SEQUENCE LISTING (1 sequence)

SEQ ID NO: 1 - DNA, 16 bp, synthetic construct
  source 1..16: mol_type = other DNA
actgttacgagtcgat

(19) United States
(12) Patent Application Publication
(10) Pub. No.: US 2025/0245870 A1
(43) Pub. Date: Jul. 31, 2025

(54) SYNTHETIC IMAGE GENERATION AND MACHINE LEARNING ANALYSIS FOR BIOLOGICAL SUBSTANCES

(71) Applicant: MGI Tech Co., Ltd, Shenzhen (CN)

(72) Inventors: Kyle Davis, Santa Clara, CA (US); Radoje T. Drmanac, Los Altos Hills, CA (US); Christian Villarosa, San Marcos, CA (US)

(21) Appl. No.: 18/983,916

(22) Filed: Dec. 17, 2024

Related U.S. Application Data
(60) Provisional application No. 63/626,168, filed on Jan. 29, 2024.

(51) Int. Cl. G06T 11/00; G06T 7/00; G06V 10/60; G06V 20/69

(52) U.S. Cl. CPC G06T 11/00; G06T 7/0012; G06V 10/60; G06V 20/695; G06T 2207/10056; G06T 2207/20081; G06T 2207/30024

(57) ABSTRACT

Systems and methods for synthetic image generation and machine learning analysis for biological substances. In one example, a set of real microscopy images representing objects of a biological substance is received. Each object corresponds to one or more pixels of the real microscopy image. Features of the real microscopy image and the objects are accessed and a set of synthetic microscopy images representing the objects of the biological substance is generated based on the features. In another example, a set of synthetic microscopy images is generated using seed intensities and features extracted or known from a set of real microscopy images. A set of seed images for the synthetic microscopy images is generated from the seed intensities. A trained machine learning model is generated to generate intensity values for additional real microscopy images using the synthetic microscopy images and the seed images as training data.

### CROSS-REFERENCES TO RELATED APPLICATIONS

The present application claims the benefit of and the priority to U.S. Provisional Application No. 63/626,168, filed on Jan. 29, 2024, the entire contents of which are herein incorporated by reference in their entireties for all purposes.

### REFERENCE TO AN ELECTRONIC SEQUENCE LISTING

The contents of the electronic sequence listing (106340-1473797-5123-US SL.xml; Size: 2,037 bytes; and Date of Creation: Dec. 12, 2024) is herein incorporated by reference in its entirety.

### FIELD

Imaging systems and methods, such as imaging systems and methods for synthetic image generation and machine learning analysis for biological substances.

### BACKGROUND

Various technologies have been developed to analyze biological substances such as tissue cells, DNA, biological liquids, etc. One of these technologies is sequencing, which itself includes different technologies distinguished by the target of their investigations such as individual strains (bacterial, viral, etc.) or a complete population, either DNA or RNA, and either all material present or just specific targeted regions. In genetics, the term sequencing refers to methods for determining a primary structure or sequence of a biopolymer, including a nucleic acid (e.g., DNA, RNA etc.). More specifically, DNA and RNA sequencing is the process of determining an order of nucleotide bases (e.g., adenine, guanine, cytosine and thymine) in a given nucleic acid fragment. Such sequencing methods commonly include calling a base at a position in a nucleic acid fragment, where the called base is used to determine a sequence for the nucleic acid.

When sequencing target nucleic acids, for example, the process typically includes extracting and fragmenting target nucleic acids from a sample. The fragmented nucleic acids are used to produce target nucleic acid templates that will generally include one or more adapters. The target nucleic acid templates may be subjected to amplification methods, such as bridge amplification to replicate and generate copies of the target nucleic acids. Sequencing applications are then performed on the copies target nucleic acids.

In traditional Sanger sequencing, each type of nucleotide (e.g., A, T, C, and G) is labeled with a different fluorescent dye. In next generation sequencing (NGS) technologies (e.g., DNA nanoball sequencing), fluorescently labeled reversible terminators are used. The nucleic acid fragments are then subjected to a sequencing reaction where new nucleic acid strands are synthesized. During this synthesis, the fluorescently labeled bases are incorporated into the growing nucleic acid chain. In Sanger sequencing, the nucleic acid fragments are separated by size using gel electrophoresis. In NGS, the fragments are often attached to a solid surface (such as a flow cell), and the sequencing reaction occurs in a massively parallel fashion. After the sequencing reaction, the fluorescence signals from the labeled bases are detected by an imaging system. Each base emits light at a characteristic wavelength due to its specific fluorescent label. The order of the bases in the sequence is determined by analyzing the fluorescence signals in the obtained images. The color and intensity of the fluorescence at each position correspond to the type of base incorporated into the sequence.

An intensity value (e.g., a fluorescence signal) corresponding to a base that is incorporated into a nucleic acid sequence at a particular position can indicate the base at that position. For example, four different types of fluorescence may be used, corresponding to the four types of bases to be identified. The nucleic acid sequences are amenable to relatively inexpensive and efficient imaging techniques in which the nucleic acid sequences are captured in four color images, one for each type of fluorescence used. The four images can then be processed through software to extract intensity information. The intensity value for a target nucleic acid template can correspond to one pixel or multiple pixels of an image, or there can be multiple templates for a pixel (i.e., more than one template per pixel). Regardless, an intensity value for each of the four bases can be assigned to a template. The images and template are processed through specialized software to convert the fluorescence signals into a readable nucleic acid sequence. The pixel intensity at each position corresponds to the type and quantity of each labeled base, allowing for the identification of the nucleic acid sequence. However, microscopy images may have a low resolution or have a high spot density, resulting in inaccurate intensity value extractions. Additionally, the biochemistry of the sequencing process can cause artifacts, and the intensity signals can vary significantly from one position and template to another, and from sample to sample.

Accordingly, it would be desirable to provide improved methods and systems for intensity extraction and base-calling.

### SUMMARY

In one example, a method involves receiving a set of real microscopy images representing a plurality of objects of a biological substance, each object of the plurality of objects corresponding to one or more pixels of the set of real microscopy images; accessing a plurality of features of the set of real microscopy images and the plurality of objects; and generating, based on the plurality of features, one or more synthetic microscopy images representing the plurality of objects of the biological substance.

In some embodiments, the method further involves performing a simulation of sequencing biochemistry for the biological substance. The simulation is configured to receive the plurality of features of a real microscopy image of the set of real microscopy images as an input. The method further involves determining, based on the input, a seed intensity for each object of the plurality of objects of the real microscopy image. The seed intensity corresponds to a signal volume for the object.

In some embodiments, the method further involves generating a seed image based on the seed intensity for each object of the plurality of objects. Each pixel in the seed image represents the signal volume for the object.

In some embodiments, generating the one or more synthetic microscopy images involves generating a point spread function for the plurality of objects of the real microscopy image based on the plurality of features; determining a signal distribution over a plurality of pixels by aggregating the point spread function and the seed intensity for each object; and generating a synthetic image of the one or more synthetic microscopy images based on the signal distribution over the plurality of pixels and the plurality of features.

In some embodiments, the method further involves generating a trained machine learning model by training a machine learning model to generate intensity values for additional real microscopy images using the one or more synthetic microscopy images and a set of corresponding seed images as training data.

In some embodiments, the biological substance comprises a DNA array, an oligo array, a biological tissue, or an array of cells.

In some embodiments, the biological substance comprises a DNA array and the plurality of objects comprises a plurality of DNA nanoballs.

In some embodiments, the one or more synthetic microscopy images have substantially similar features to those of the set of real microscopy images.

In one example, a method involves generating a set of synthetic microscopy images using seed intensities and features extracted or known from a set of real microscopy images, each synthetic microscopy image representing a plurality of objects of a biological substance; generating a set of seed images from the seed intensities, where each seed image corresponds to a synthetic microscopy image of the set of synthetic microscopy images, and where each pixel in the seed image represents a signal volume for an object of the plurality of objects; and generating a trained machine learning model by training a machine learning model to generate intensity values for additional real microscopy images using the set of synthetic microscopy images and the set of seed images as training data.

In some embodiments, the method further involves inputting a real microscopy image into the trained machine learning model. The real microscopy image depicts an additional plurality of objects. The method can further involve receiving, from the trained machine learning model, an output representing a seed intensity for each object of the additional plurality of objects in the real microscopy image and generating a simulated microscopy image corresponding to the real microscopy image based on the output.

In some embodiments, the method further involves determining a difference between the real microscopy image and the simulated microscopy image.

In some embodiments, the method further involves in response to determining the difference, determining a set of features to use to generate subsequent simulated microscopy images.

In some embodiments, the trained machine learning model is a first trained machine learning model, and the method further involves in response to determining the difference, inputting the simulated microscopy image into a second trained machine learning model, and receiving, from the second trained machine learning model, a result of an adjusted simulated microscopy image corresponding to the real microscopy image.

In some embodiments, generating the set of synthetic microscopy images involves: receiving a set of real microscopy images representing a plurality of objects of the biological substance, each object of the plurality of objects corresponding to one or more pixels of the set of real microscopy images; accessing a plurality of features of the set of real microscopy images and the plurality of objects; and generating, based on the plurality of features, one or more synthetic microscopy images representing the plurality of objects of the biological substance.

In some embodiments, the method involves performing a simulation of sequencing biochemistry for the biological substance. The simulation is configured to receive the plurality of features of a real microscopy image of the set of real microscopy images as an input. The method further involves determining, based on the input, a seed intensity for each object of the plurality of objects of the real microscopy image. The seed intensity corresponds to a signal volume for the object.

In some embodiments, the method further involves generating a seed image based on the seed intensity for each object of the plurality of objects. Each pixel in the seed image represents the signal volume for the object.

In some embodiments, generating the one or more synthetic microscopy images involves: generating a point spread function for the plurality of objects of the real microscopy image based on the plurality of features; determining a signal distribution over a plurality of pixels by aggregating the point spread function and the seed intensity for each object; and generating a synthetic image of the one or more synthetic microscopy images based on the signal distribution over the plurality of pixels and the plurality of features.

In some embodiments, the biological substance comprises a DNA array, an oligo array, a biological tissue, or an array of cells.

In some embodiments, the biological substance comprises a DNA array and the plurality of objects comprises a plurality of DNA nanoballs.

In some embodiments, the machine learning model comprises a residual channel attention network.

In one example, a method for training a machine learning model involves: providing a set of real microscopy sequencing images of DNA or DNA nanoball array-based sequencing of a reference sequence, the set of real microscopy images being representative of a sequencing process; providing a plurality of base calls for the set of real microscopy sequencing images determined using a mapping to the reference sequence or a plurality of sequence barcodes; and generating a trained machine learning model that generates base call probabilities from sequencing images by using the set of real microscopy sequencing images and the plurality of base calls as training data.

In one example, a method involves: receiving a set of real microscopy images depicting a plurality of objects of a biological substance, the set of real microscopy images corresponding to different labeled bases in a DNA sequence; converting, by a first trained machine learning model the set of real microscopy images into an array of base-call probabilities for each object of the plurality of objects; and determining, by a second trained machine learning model, a base-call for each object of the plurality of objects in each real microscopy image of the set of real microscopy images based on the array of base-call probabilities and a sequence context of the base-call for each object.

In some embodiments, the first trained machine learning model and the second trained machine learning model are a same or different machine learning model.

In some implementations, a computer-program product is provided that is tangibly embodied in a non-transitory machine-readable medium, including instructions configured to cause one or more data processors to perform the computer-implemented methods or operation of any of the preceding claims.

In some implementations, a system is provided comprising: one or more data processors; and a non-transitory computer readable medium storing instructions which, when executed on the one or more data processors, cause the one or more data processors to perform the computer-implemented method or operations of any of the preceding claims.

### BRIEF DESCRIPTION OF FIGURES

FIG. 1 is a block diagram illustrating an example system for synthetic image generation and machine learning analysis according to various embodiments.

FIG. 2 (SEQ ID NO:1) illustrates an example of sampling a real sequence from a reference genome according to various embodiments.

FIG. 3 (SEQ ID NO:1) illustrates an example of a simulation of phasing events that occur during sequencing according to various embodiments.

FIG. 4 (SEQ ID NO:1) illustrates an example of a simulation of incorporation events that occur during sequencing according to various embodiments.

FIG. 5 (SEQ ID NO:1) illustrates an example of phasing through multiple cycles according to various embodiments.

FIG. 6 illustrates example point spread function tables according to various embodiments.

FIG. 7 illustrates an example point spread array according to various embodiments.

FIG. 8 illustrates an example background distribution for a point spread array according to various embodiments.

FIG. 9 illustrates additional example background distributions for a point spread array according to various embodiments.

FIG. 10 illustrates an example synthetic microscopy image according to various embodiments.

FIG. 11 illustrates an example of images throughout synthetic image generation and machine learning analysis according to various embodiments.

FIG. 12 illustrates an example of a machine learning model 800 for predicting a base-call for a DNA nanoball according to various embodiments.

FIG. 13 is a flowchart of a method for generating synthetic microscopy images according to various embodiments.

FIG. 14 is a flowchart of a method for developing an intensity extraction model according to various embodiments.

FIG. 15 is a flowchart of a method for using a base-calling model according to various embodiments.

FIG. 16 shows a block diagram of an example computer system usable with system and methods according to various embodiments.

FIG. 17 shows example results of using a machine learning model for intensity extraction according to various embodiments.

### DETAILED DESCRIPTION

### Introduction

Embodiments described herein pertain in general to the use of machine learning to synthetically generate a single-pixel “image” with each pixel corresponding to the accurate intensity of a DNA spot from a real digital image. These are final intensities without signal bleed from neighboring DNA-spots and background in an array, such as a DNA or a DNA nanoball (DNB) array. As such, there may be no DNA or DNB wobbling. The machine learning based techniques allow for the precise fluorescent intensities to be extracted per each nucleic acid strand or DNB. Base calls may then be determined using algorithmic approaches and/or additional machine learning based techniques, and a quality score is calculated from the final intensities. The machine learning based techniques described herein are especially advantageous for low resolution and/or high spot density images as can be observed in sequencing technologies such as DNA nanoball sequencing. In this instance, machine learning models can be trained to generate higher resolution images or single-pixel images with subpixel resolution relative to original images for improved intensity extraction and ultimately base call determination. Although, the machine learning based techniques are described herein with particularity to DNA/DNB sequencing it should be understood that these techniques are generally applicable for any type of technology that generates images (e.g., whole slide images, tissues sections, or random cell microscopy).

However, these machine learning based techniques require single-pixel “images” with accurate intensities, or high resolution images for other applications, to train the machine learning models. Disclosed herein are two solutions to generate these accurate intensities or high-resolution images for training the machine learning models.

The first solution pertains to high resolution imaging and includes generating high resolution images through the use of an advanced high NA imager, or through using super-resolution based imaging that uses multiple images of the same array at lower resolution to reconstruct the image at high resolution. The extracted final intensities are then used from these images as the ground truth values for training of the machine learning models together with real low-resolution images.

The second solution pertains to realistic simulated images and includes simulating realistic images as an array of overlapping signal distributions. The spacing between the center of the distributions in the array is reflective of the spacing between DNBs in real images. The signal distributions are also simulated at the nanometer scale allowing for the pixel size of the image to be scaled and reflect the pixel size of real images. The shape of these signal distributions is determined by factors that are measured from real images. The signal volume for each distribution is generated from a simulation of the sequencing biochemistry, giving each distribution a unique value that corresponds with the distribution of DNB signal across the image that is typically seen in real data. The signal volume for each DNB is saved as a seed image, with each pixel in the seed image representing the corresponding DNBs signal volume. The seed image is then be used as the ground truth values for training the machine learning models on the simulated images.

Once the machine learning models are trained using either solution to generate a single-pixel “image” with each pixel corresponding to the accurate intensity of a DNA spot from a real digital image, one or more additional machine learning models (e.g., convolutional neural networks) can be implemented to directly analyze and predict the base call for each DNB from a single-pixel “image”. In some instances, these techniques take advantage of two machine learning models that are trained on the same data to produce highly accurate base-calls. The first machine learning model takes four images corresponding to the different labeled bases and converts them into an array of probabilities of the likely base to be called for each DNB. The second machine learning model uses the base-call probability of multiple cycles to further enhance the base-calling accuracy by providing the sequence context of the base-call for each DNB. These machine learning models can be combined into a single pipeline that reconfigures the output of multiple cycles from the first model to act as the input for the second model. The result is an array of refined base-call probabilities that can be used to calculate the correct base-call as well as quality score metrics for each DNB. In another embodiment, a single machine learning model (e.g., a single CNN) can be trained that performs both steps of the pipeline.

Intensity Extraction from DNA/DNB-Array and Analysis Thereof Using Machine Learning

Embodiments described herein provide synthetic image generation and intensity extraction for images (e.g., microscopy images) in a sequencing process. A machine learning model can be developed to determine intensity values of objects depicted in real images. The machine learning model can be trained on synthetic images in which seed intensity values for objects depicted in the synthetic images are known. Synthetic images are created by means of mathematical modelling computations of compiled data rather than by the more traditional photographic process of generating real images using light waves focused through cameras or other optical instruments. Conventionally, intensity values in real images may be difficult to determine due to the images having a low resolution or the objects being of a high density in the images. However, since the synthetic images are generated to substantially mimic real images, the machine learning model learns during training how to interpret images having a low resolution or high density of objects, which enables intensity values to be accurately extracted from real microscopy images during inference.

The embodiments may be applied to various microscopy images, such as those depicting tissue cells, DNA arrays, oligo arrays, etc. In an example in which embodiments are applied to a sequencing technique for DNBs, intensity values at each location in an image associated with a DNBs correspond to a base of the DNB for that cycle. So, in a synthesized image, each pixel corresponds to an accurate intensity of a DNA spot from a real image. These pixels are final intensities with minimal signal bleed from neighboring DNA-spots and a background in a perfect array with no DNA wobbling.

The embodiments also involve a machine learning model that can directly predict a base-call for a DNA nanoball. The machine learning model receives four input images at a first machine learning model that generates an array of probabilities of the base for each DNA nanoball. A second machine learning model receives arrays for multiple cycles and adjusts the probabilities of the base for each DNA nanoball. In this case, the machine learning model still uses microscopy images to generate a base-call prediction but bypasses a seed intensity calculation for the images.

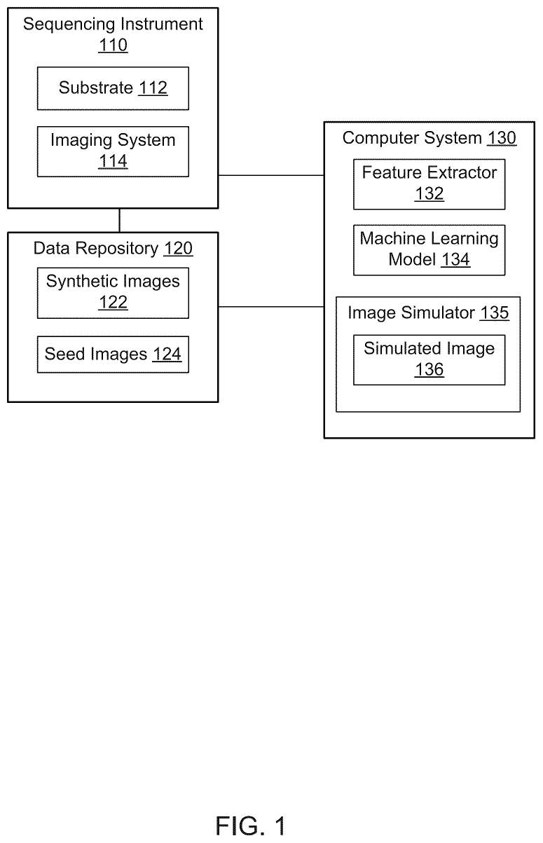

FIG. 1 is a block diagram illustrating an example system 100 for synthetic image generation and machine learning analysis according to one embodiment. In this embodiment, system 100 may include a sequencing instrument 110 and a computer system 130. The computer system 130 is connected to the sequencing instrument 110 via a direct wired or wireless connection or via a high-speed local area network (not shown). The sequencing instrument 110 includes primary sub-systems, such as a substrate 112 for holding a biological substance and an imaging system 114.

The computer system 130 includes typical hardware components (not shown) including, one or more processors, input devices (e.g., keyboard, pointing device, etc.), and output devices (e.g., a display device and the like). The computer system further includes computer-readable/writable media, e.g., memory and storage devices (e.g., flash memory, a hard drive, an optical disk drive, a magnetic disk drive, and the like) containing computer instructions that implement the functionality disclosed when executed by the processors. The computer system 130 may further include software and/or hardware for controlling the sequencing instrument 110.

Sequencing can operate on input biological substances, which may be obtained by extracting a sample from a target organism. For example, the biological substance may be a DNA array, an oligo array, an array of cells, biological tissue, biological liquids, etc. In some instances, the sequencing performed is DNB sequencing, which is a high throughput sequencing technology that can be used to determine the entire genomic sequence of the target organism. Created during library prep, compact DNBs are captured onto one or more substrates 112, and the substrates 112 are then inserted into the sequencing instrument 110. The substrate 112 may be either un-patterned or patterned. In the un-patterned embodiment, samples of biological substance may each be deposited in discrete locations on the substrate 112, but the locations need not be fixed. The substrate 112 is then loaded into the sequencing instrument 110 and the sequence is read with fluorescent probes recognizing the DNA or RNA bases.

More specifically, DNB sequencing involves isolating DNA that is to be sequenced and shearing it into small 100-350 base pair (bp) fragments. Isolation includes lysing cells and extracting the DNA from the cell lysate. The high-molecular-weight DNA, often several megabase pairs long, are then fragmented by physical or enzymatic methods to break the DNA double-strands at random intervals. For small RNA sequencing, selection of the ideal fragment lengths for sequencing may be performed by gel electrophoresis, whereas for DNA sequencing of larger fragments, DNA fragments may be separated by bead-based size selection.

DNB sequencing further involves ligating adapter sequences to the fragments and circularizing the fragments. Adapter sequences may be attached to the unknown DNA so that DNA segments with known sequences flank the unknown DNA. In the first round of adapter ligation, right (Ad153_right) and left (Ad153_left) adapters may be attached to the right and left flanks of the fragmented DNA, and the DNA is amplified by PCR. A splint oligo can then hybridize to the ends of the fragments which are ligated to form a circle. An exonuclease is added to remove all remaining linear single-stranded and double-stranded DNA products. The result is a completed single-stranded circular DNA template.

Once a single-stranded circular DNA template is generated, containing sample DNA that is ligated to two unique adapter sequences, the full sequence is amplified into a long string of DNA. This is accomplished by rolling circle replication with a polymerase such as the Phi 29 DNA polymerase which binds and replicates the DNA template. The newly synthesized strand is released from the circular template, resulting in a long single-stranded DNA comprising several head-to-tail copies of the circular template. The resulting nanoparticle self-assembles into a tight ball of DNA (DNB) approximately 300 nanometers (nm) across. DNBs remain separated from each other because they are negatively charged and naturally repel each other, reducing tangling between different single stranded DNA lengths.

To obtain the sequence of the nucleic acids, the DNBs are attached to a patterned array flow cell (e.g., substrate 112). The flow cell may be a silicon wafer coated with silicon dioxide, titanium, hexamethyldisilazane (HMDS), and a photoresist material. The DNA nanoballs are added to the flow cell and selectively bind to the positively-charged aminosilane in a highly ordered pattern, allowing a very high density of DNA nanoballs to be sequenced. From there the flow cells are loaded in the sequencing instrument 110, combinatorial probe-anchor synthesis (cPAS) chemistry is used to hybridize sequencing primers to the DNBs and fluorescently labeled reversibly terminated probes are incorporated by DNA polymerase in consecutive sequencing cycles.

After each DNA nucleotide incorporation step, the fluorescent probes are excited by laser light and the imaging system 114 captures an image of the biological substance on the substrate 112. In the particular instance where the biological substance is DNA organized in an array of DNBs, the image represents the DNBs. From the image, the genomic sequence of each DNA nanoball may be determined. An intensity value (e.g., a fluorescence signal) corresponding to a base that is incorporated into a nucleic acid at a particular position can indicate the base at that position. Since the intensity value may be difficult to extract from the image, a machine learning model 134 may be developed for predicting intensity values of real microscopy images as described in further detail below.

In order to assist with development of the machine learning model 134, synthetic microscopy images with known intensity values may be generated based on real microscopy images. Each synthetic microscopy image can mimic a real microscopy image. In addition, multiple synthetic microscopy images may be generated from a single real microscopy image. The synthetic microscopy images and known intensity values can then be used to train the machine learning model 134 to extract intensity values from real microscopy images.

In general, synthetic images are simulated with several quality defining features that are predefined, measured, or estimated from real images such that the synthetic images are realistic representations of real images. This feature set includes but is not limited to: spacing between objects (DNBs), nanometer size of the pixels, emission wavelength of fluorophores, effective NA of the combination of the imaging instrument along with slide solution, degradation of signal spread along an axis due to imaging process, variation in the number of photons emitted per fluorophore, variation in the number of photons registered per pixel, as well as signal independent and dependent background average and variation, and other sufficiently reproducible non-uniformities and artifacts. So, each synthetic microscopy image has substantially similar features to that of a realistic microscopy image. As used herein, the terms “similarly”, “substantially,” “approximately” and “about” are defined as being largely but not necessarily wholly what is specified (and include wholly what is specified) as understood by one of ordinary skill in the art. In any disclosed embodiment, the term “similarly”, “substantially,” “approximately,” or “about” may be substituted with “within [a percentage] of” what is specified, where the percentage includes 0.1, 1, 5, and 10 percent.

In one embodiment, to generate a synthetic microscopy image, the computer system 130 generates high resolution images through the use of an advanced high numerical aperture imager, or through using super-resolution-based imaging that uses multiple images of the same substrate at lower resolution to reconstruct the image at high resolution. The extracted final intensities from these images can be used as ground truth values for training purposes together with real low-resolution images.

As another example, to generate synthetic microscopy images, the computer system 130 can receive a set of real microscopy images representing objects (e.g., DNA nanoballs, tissue cells, etc.) of a biological substance. A feature extractor 132 of the computer system 130 can then access features of the real microscopy images and the objects. Accessing the features may involve actively determining or importing features from a stored location. The features can include physical characteristics of the objects and imaging characteristics of the real microscopy images. The physical characteristics can include a size of the objects (e.g., DNA nanoball diameter), a distance between neighboring objects (nm pitch), a distribution for a number of photons emitted from each dye, etc. The imaging characteristics can include a pixel distance between objects (pixel pitch) or pixels per object, a pixel photon registration (shot noise), a signal independent background and variation, a signal dependent background and variation, optical aberrations, focus precision n position effects, non-uniformity of illumination, etc.

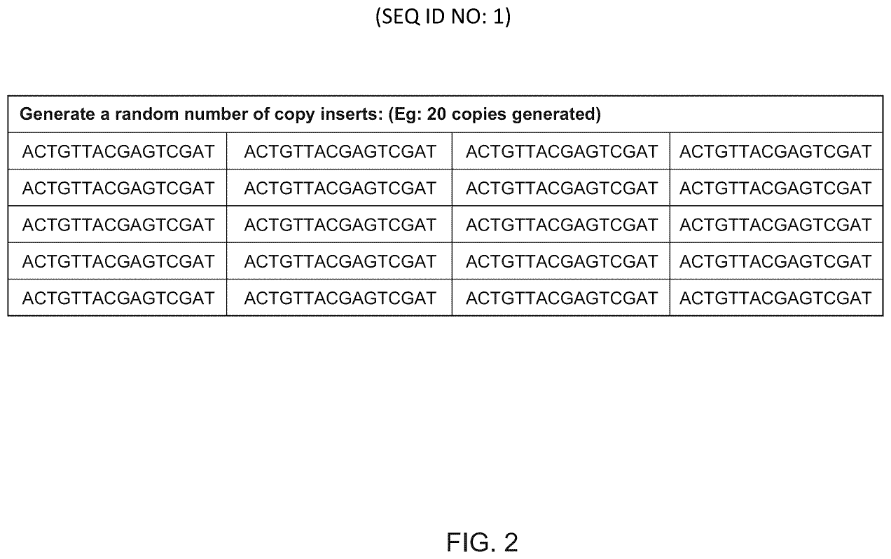

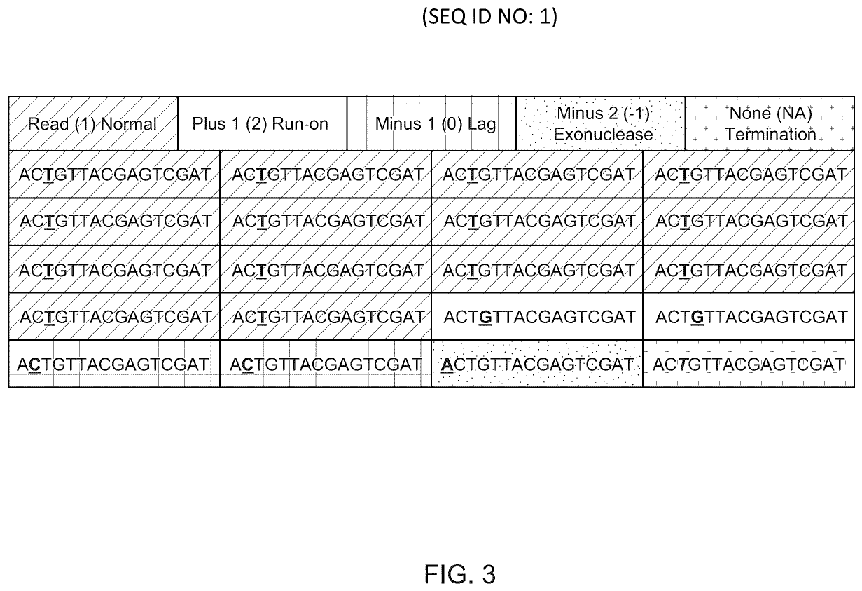

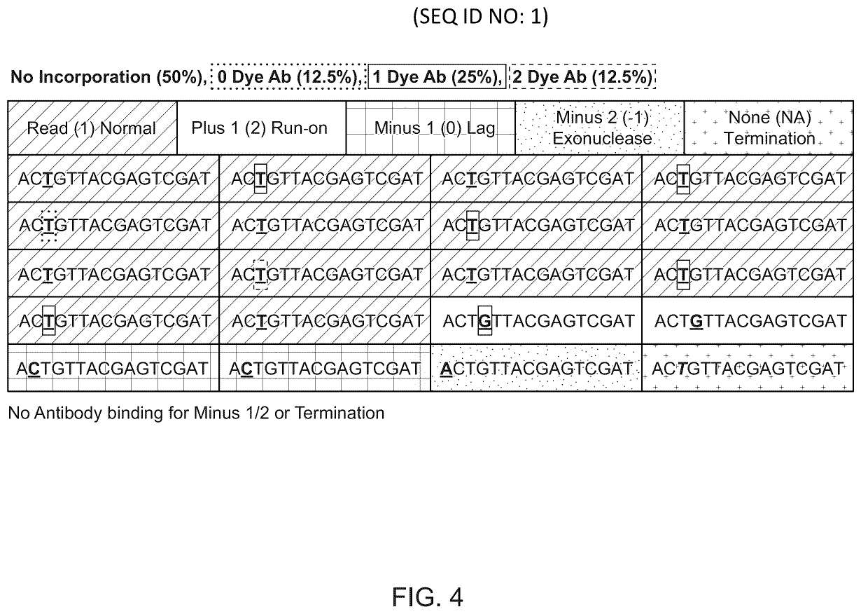

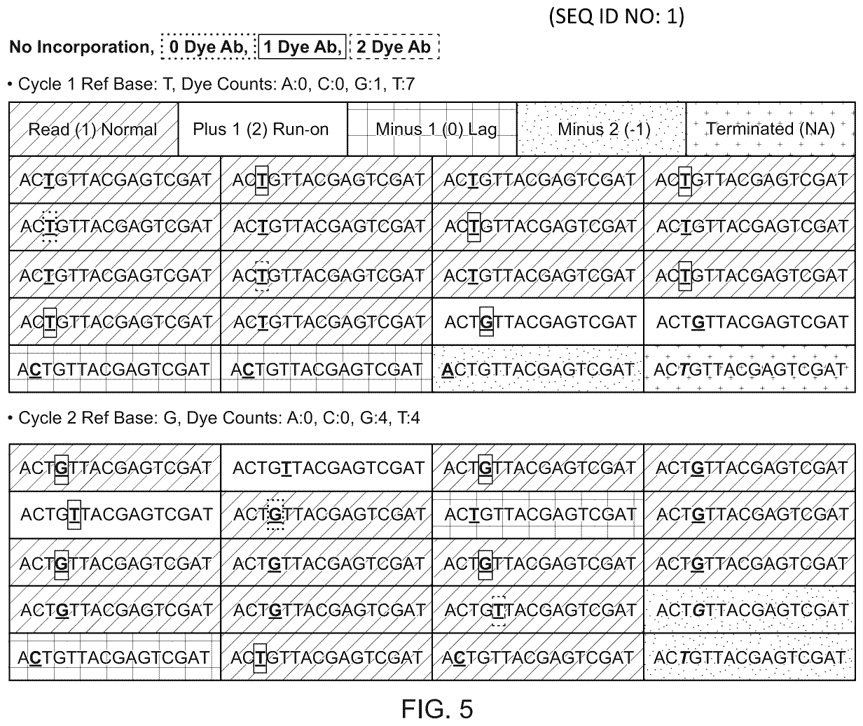

In addition, the computer system 130 can perform a simulation of sequencing biochemistry to model biochemical events that can occur during sequencing to ultimately generate the synthetic microscopy image. The simulation can receive the features for a real microscopy image as an input. As shown in FIG. 2, the simulation can involve generating a distribution of copy numbers for different objects of real sequences sampled from a reference genome. The computer system 130 can assign a copy number for an object from a realistic distribution by choosing a copy number from a normal distribution or by calculating a copy number from the combination of a random insert size and random object mass (e.g., in kilobases). As an example, the random sequence may be ACTGTTACGAGTCGAT (SEQ ID NO:1). In addition, the simulation can further involve modeling random phasing events (e.g., lag, run-on, termination, and exonuclease activity), which is shown in FIG. 3. The read position is at position three, corresponding to the first T. The copy breakdown of FIG. 3 is fourteen in-phase, two plus one run-ons, two minus one lags, one minus two exonuclease activity, and one termination. The simulation may also model incorporation events (e.g., antibody incorporation, dyes per antibody distribution, and unwashed dyes), as shown in FIG. 4. A Poisson distribution of dyers per antibody is set as a list where each element is the average number of dyes per antibody for the respective channel (e.g., A, C, T, and G). A percentage distribution has key value pairs of (percentage of antibodies:number of dyes per antibody). As an example, (0.25:0, 0.5:1, 0.25:2) corresponds to 25% of antibodies having zero dyes, 50% of antibodies having one dye, and 25% of antibodies having two dyes. In FIG. 4, 50% have no incorporation, 12.5% of antibodies have zero dyes, 25% of antibodies have one dye, and 12.5% of antibodies have two dyes. There is not antibody binding for minus one, minus two, or termination events. The phasing is repeated through antibody incorporation for a specified number of cycles. The number of dyes that are present in each channel at each cycle can be aggregated and these values can represent the seed intensity for an object, as shown in FIG. 5. So, the output of the sequencing biochemistry is the seed intensities of the objects of a real microscopy image.

Generating the synthetic microscopy images can also involve the computer system 130 generating a point spread function for the objects of a real microscopy image based on features of the real microscopy image. The point spread function can be generated using either a Bessel function (e.g., Airy Disk) or a Gaussian approximation of the Airy Disk. The point spread function can result in a point spread function table at the nanometer scale. The point spread function for a full object can be comprised of the aggregation of multiple point spread functions within the object diameter area. The Gaussian function is expressed as:

$$I ( x ) = {I}_{0} * exp ( - {( \frac{f ( x )}{1.889} )}^{2} ) \qquad ( 1 )$$

$$f ( x ) = \frac{2 π * NA * ( x - {x}_{0} )}{λ} \qquad ( 2 )$$

where I₀ is the intensity value of an object, NA is the numerical aperture and is inversely related with the spread of the function, λ is the emission wavelength of light and is directly related with the spread, x₀ is the center point of the point spread function, and x is the point in the real microscopy image.

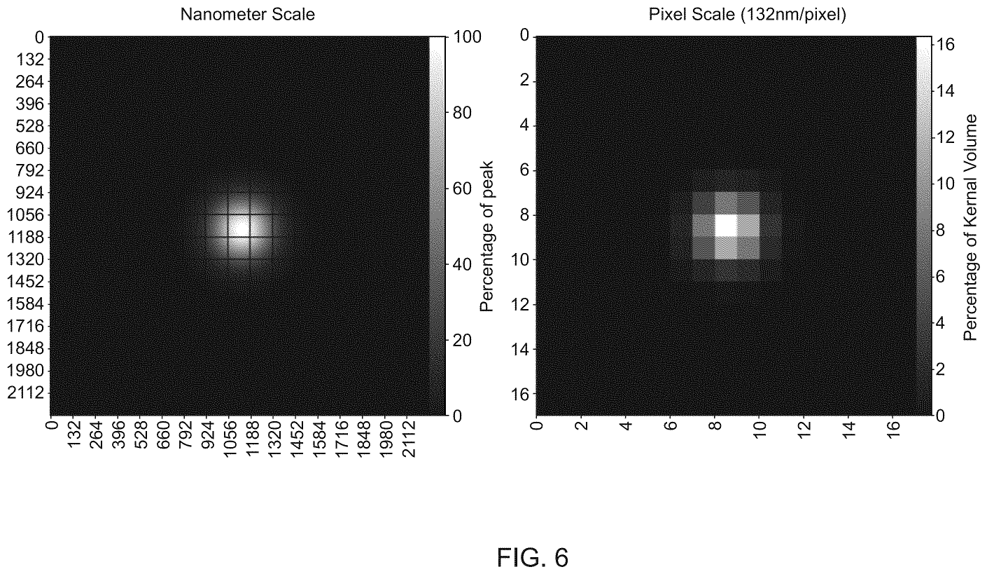

The computer system 130 can integrate the point spread function table at the nanometer scale into the appropriate pixel sizes to generate a point spread function table at a pixel scale, as shown in FIG. 6. Integration may be done at 0.01 subpixel reading frames. In FIG. 6, the NA is 0.9, the wavelength is 500 nm, there are 150 nm/pixel, and the object size is 150 nm. The point spread function table at the pixel scale can be normalized to 1 so that a seed intensity for a specific object corresponds to the volume of the point spread function. The seed intensity corresponds to a signal volume for the object. The computer system 130 may then multiply the seed intensities by a scalar from a normal distribution representing variation in the quantum yield of the dyes. These values (or the seed intensities themselves) can then be aggregated with the point spread function table at the pixel scale to generate a signal distribution of the object over the pixels. The aggregation may involve multiplying the seed intensities for each object and the point spread function.

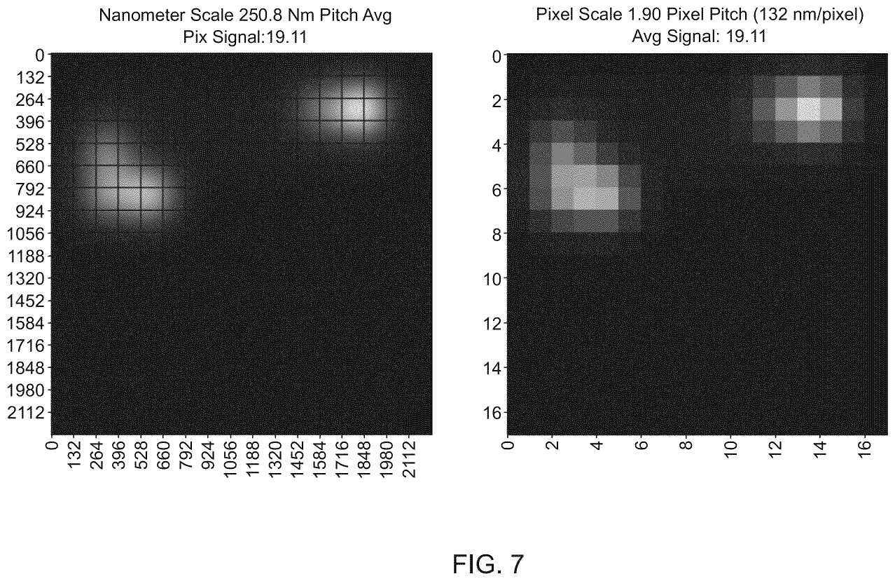

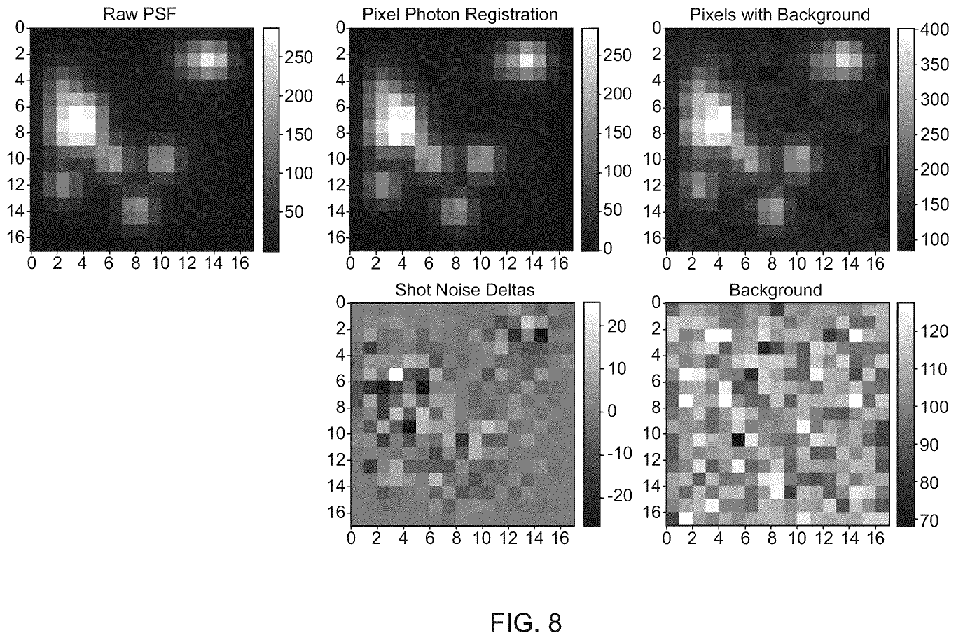

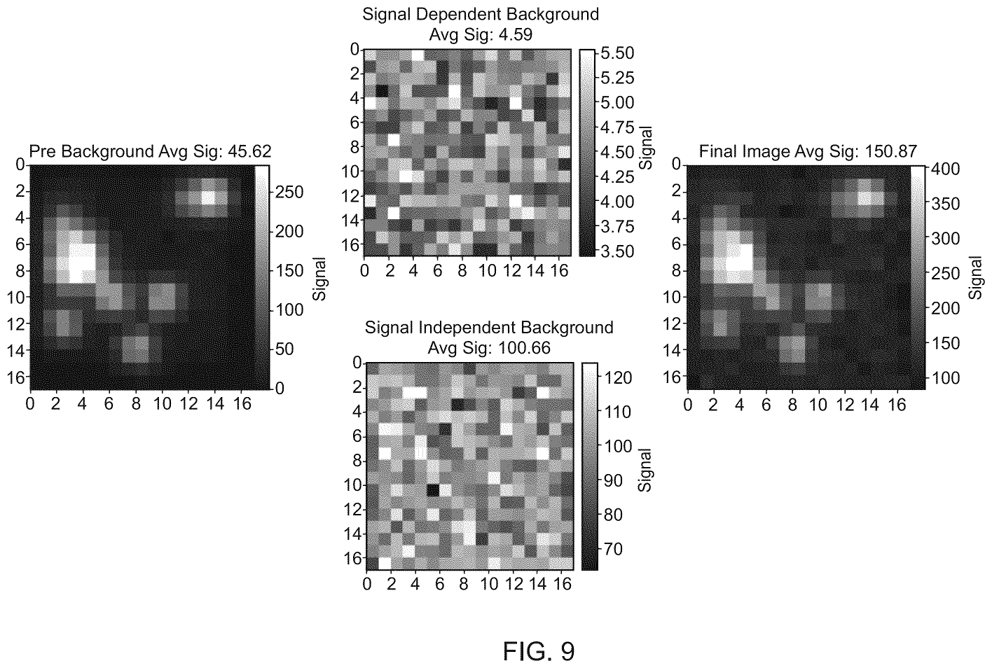

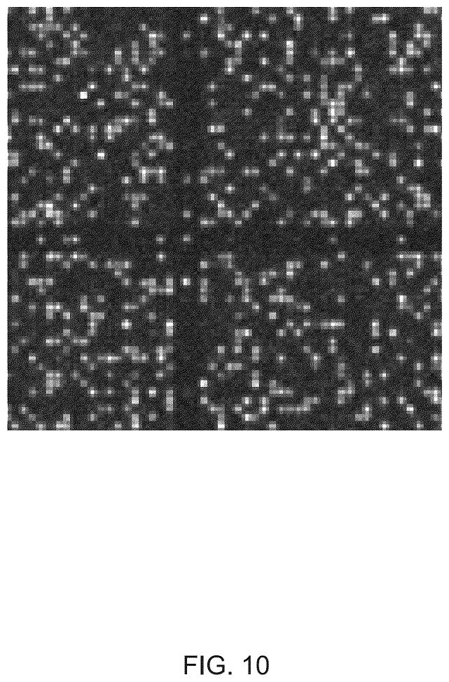

The computer system 130 can place multiple signal distributions onto an image as in an array, as shown in FIG. 6. The signal distributions can be spaced as designated by the features of the real microscopy image. The spacing (pitch) between objects can be predefined. In FIG. 7, the pitch is 300 nm and 1.9 pixels with an NA of 0.9. Background distributions can then be applied to the object point spread function arrays, as shown in FIG. 8. In an example, a Poisson distribution can be applied to pixels of the image to represent shot noise. The Poisson distribution corresponds with the number of photons each pixel registers. Two additional normal distributions can also be applied to the pixels in the image to represent signal independent background and signal dependent background, as shown in FIG. 9. The signal independent background is the normal distribution based on a scalar value that does not scale with the signal. The signal dependent background is the normal distribution based on a scalar value that scales with the 80^{th} percentile value of the pre-background image. The computer system 130 then uses the features to generate one or more synthetic microscopy images representing the objects of the biological substance. The synthetic microscopy images can substantially mimic the real microscopy image, but the intensity values of the objects in the synthetic microscopy image are known. An example of a synthetic microscopy image is illustrated in FIG. 10.

So, in general, realistic images can be simulated as an array of overlapping signal distributions. The spacing between the center of the distributions in the array can be reflective of the spacing between the objects in real microscopy image. The signal distributions can also be simulated at the nanometer scale allowing the pixel size of the synthetic image to be scaled to reflect the pixel size of real image. Signal distribution parameters can include NA, wavelength, etc. The shape (e.g., circular, elliptical where the X and Y positions have different NA values, etc.) of these signal distributions can be determined by factors that are measured from the real image. In addition, the signal volume for each distribution can be generated from a simulation of the sequencing biochemistry, giving each distribution a unique value that corresponds with the distribution of object signal across the image that is in real data. A seed image can be generated based on the signal volume (seed intensity) for each object, with each pixel in the seed image representing the corresponding object signal volume. The single pixel intensities can be at a subpixel resolution relative to the real microscopy images.

Synthetic images 122 can be generated for various real microscopy images. The synthetic images 122, along with their corresponding seed images 124, can be used to train the machine learning model 134 to determine intensity values of real microscopy images. The machine learning model 134 can use a residual channel attention network (RCAN) architecture with convolutional neural network (CNN) layers. The machine learning model 134 can also be any other suitable machine learning model trained for providing a prediction, such as a generative machine learning model.

To train the machine learning model 134, the computer system 130 can acquire training data from a data repository 120. The training data can include the synthetic images 122 and the seed images 124. In some instances, portions of the training data (e.g., real images) may be augmented with the synthetic images 122 and the seed images 124. In other instances, the training data is entirely made up of the synthetic images 122 and the seed images 124. The computer system 130 uses a trainer and validator as part of a machine learning operationalization framework comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for the machine learning model 134. Specifically, the trainer performs iterative operations of training that involve inputting portions of the training data into machine learning model 134 to find a set of model parameters (e.g., weights and/or biases) that minimizes or maximizes an objective function (e.g., a loss function, a cost function, a contrastive loss function, etc.). The objective function can be constructed to measure the difference between the outputs inferred using the machine learning model 134 and the ground truth annotated to the images using the labels. For example, for a supervised learning-based model, the goal of the training is to learn a function “h( )” (also sometimes referred to as the hypothesis function) that maps the training input space X to the target value space Y, h: X→Y, such that h(x) is a good predictor for the corresponding value of y. Various different techniques may be used to learn this hypothesis function. In some machine learning algorithms such as a neural network, this is done using back propagation. The current error is typically propagated backwards to a previous layer, where it is used to modify the weights and bias in such a way that the error is minimized or maximized. The weights are modified using the optimization function. Optimization functions usually calculate the error gradient, i.e., the partial derivative of the objective function with respect to weights, and the weights are modified in the opposite direction of the calculated error gradient. For example, techniques such as back propagation, random feedback, Direct Feedback Alignment (DFA), Indirect Feedback Alignment (IFA), Hebbian learning, and the like are used update the model parameters in such a manner as to minimize or maximize this objective function. This cycle is repeated until the minimum or maximum of the objective function is reached.

The trainer also performs the process of selecting hyperparameters, using an optimization algorithm, to find the parameters that correspond to the best fit between prediction and actual outputs. Example optimization algorithms include a stochastic gradient descent algorithm or a variant thereof such as batch gradient descent or minibatch gradient descent. The hyperparameters are settings that can be tuned or optimized to control the behavior of the machine learning model 134. Most models explicitly define hyperparameters that control different features of the models such as memory or cost of execution. However, additional hyperparameters may be defined to adapt the machine learning model 134 to a specific scenario. For example, the hyperparameters may include the number of hidden units of a model, the learning rate of a model, the convolution kernel width, the number of kernels for a model, the maximum depth of a tree in a random forest, a minimum sample split, a maximum number of leaf nodes, a minimum number of leaf nodes, and the like.

Once the set of model parameters are identified, the machine learning model 134 has been trained and the computer system 130 may perform additional processes of testing or validation using a subset of the training data. The validation process includes iterative operations of inputting the validating datasets into the machine learning model 134 using a validation technique such as K-Fold Cross-Validation, Leave-one-out Cross-Validation, Leave-one-group-out Cross-Validation, Nested Cross-Validation, or the like to fine tune the hyperparameters and ultimately find the optimal set of hyperparameters. Once the optimal set of hyperparameters are obtained, a reserved set of testing data, from the initial splitting of the training data, are input into the machine learning model 134 to obtain output (in this example, predictions as to the intensity v), and the output is evaluated versus ground truth values using correlation techniques such as Bland-Altman method and the Spearman's rank correlation coefficients and calculating performance metrics such as the error, accuracy, precision, recall, receiver operating characteristic curve (ROC), etc. The metrics may be used to analyze performance of the machine learning model 134 for providing recommendations.

Once trained, the computer system 130 can input a real microscopy image into the machine learning model 134, which generates an output representing a seed intensity for each object in the real microscopy image. From the output, an image simulator 135 can generate a simulated image 136 where each pixel of the simulated image 136 corresponds to the seed intensity of the object in the real microscopy image. That is, if the real microscopy image is of an array of DNA nanoballs, the simulated image 136 can have pixels that correspond to each DNA nanoball, and the pixel values can correspond to the seed intensities of the DNA nanoballs. As such, the machine learning model 134 allows intensity values of objects to be determined from real microscopy images. The seed intensities can be used for base calling.

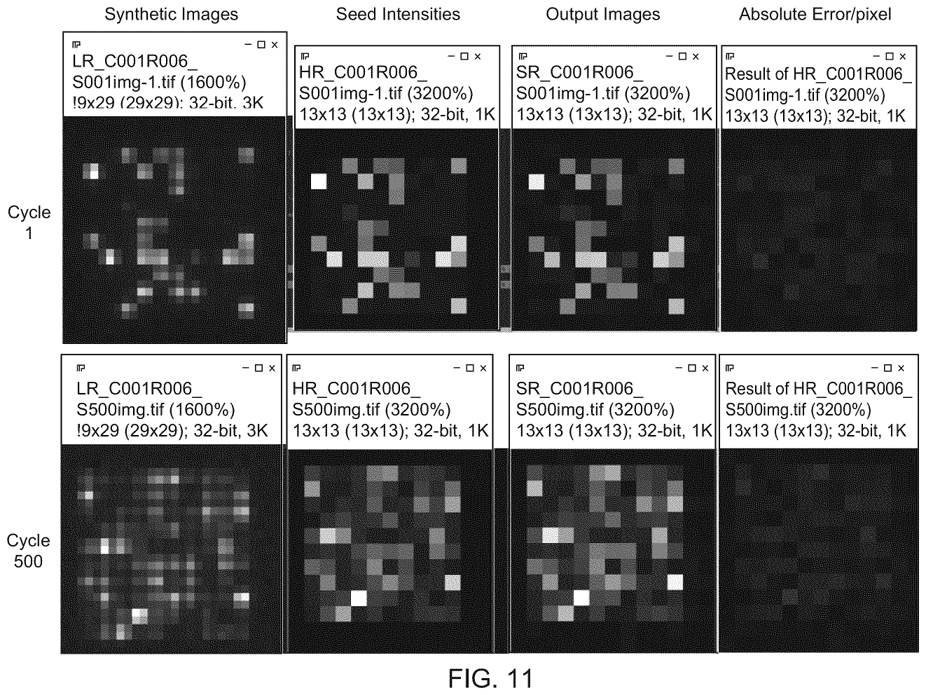

FIG. 11 illustrates an example of images throughout synthetic image generation and machine learning analysis according to one embodiment. Synthetic images generated for real microscopy images are shown for two cycles (cycle 1 and cycle 500) of DNA sequencing. In addition, seed intensity images associated with the synthetic images are also shown. The synthetic images and the seed intensity images are generated as previously described. A machine learning model (e.g., machine learning model 134 in FIG. 1) can receive the real microscopy image for each of the synthetic images and generate the simulated images showing intensity values. Images representing an absolute error for each pixel can then be generated to evaluate a performance of the model. As shown in FIG. 11, the machine learning model can accurately determine the seed intensities of objects from real microscopy images.

The computer system 130 can evaluate an accuracy of the simulated image 136 by comparing the simulated image 136 to the real microscopy image and determining differences. If a difference (e.g., an absolute error for the pixels) is determined, the computer system 130 may modify the image simulator 135 to generate more realistic images. For instance, the computer system 130 may use another machine learning model to determine a set of features to use to generate subsequent simulated microscopy images. The features may be any of the features previously described (e.g., features from a simulation of sequencing biochemistry, physical characteristics of the objects, imaging characteristics of the real microscopy image, etc.). Upon determining the set of features, the computer system 130 may update the machine learning model 134 based on the set of features.

Additionally or alternatively, the image simulator 135 may include another trained machine learning model (e.g., a CNN) that acts as a final step in image simulation. The simulated image 136 can be input into the machine learning model to generate a result of an adjusted simulated microscopy image that corresponds to the real microscopy image. The adjustments made to the simulated image 136 may be changes that the current parameter set used by the machine learning model 134 cannot account for. To train the machine learning model, simulated images can be used as input with real microscopy images being used as ground truths. Both sets of images can use the same seed intensities as ground truths from the machine learning model 134, which are extracted from the real microscopy images and used as inputs for generating the simulated images.

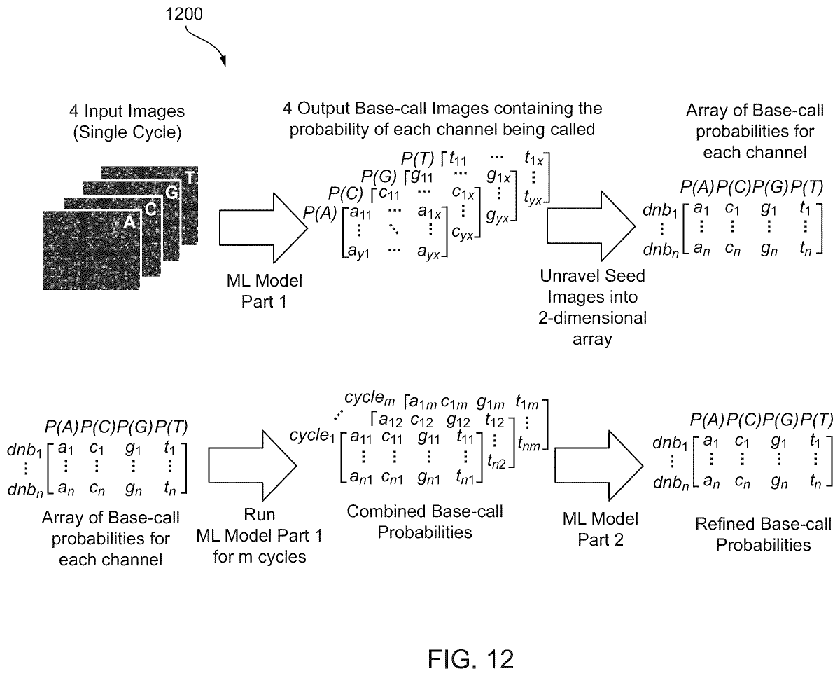

FIG. 12 illustrates an example of a machine learning model 1200 for predicting a base-call for a DNA/DNB according to one embodiment. The machine learning model 1200 may be another example of the machine learning model 134 in FIG. 1. The machine learning model 1200 can involve a machine learning model, that may be a combination of machine learning models (e.g., CNNs) trained on the same data to produce accurate base-calls. The machine learning models may be a same or different machine learning models. Once trained, a first machine learning model can receive a set of images (e.g., four images) corresponding to different labeled bases in a DNA sequence. Each image can depict a DNA array of DNA nanoballs. The first machine learning model can convert the images into an array of probabilities of the base that is likely to be called for each DNA nanoball. The second machine learning model can use the base-call probability of multiple cycles to further enhance the base-calling accuracy by providing a sequence context of the base-call for each DNA nanoball. The machine learning models may be combined into a single pipeline that reconfigures the output of multiple cycles from the first machine learning model to act as the input for the second machine learning model. The result is an array of refined base-call probabilities that can be used (e.g., by the computer system 130 in FIG. 1) to calculate the correct base-call as well as quality score metrics for each DNA nanoball. In another embodiment, one model can be trained that performs both steps.

Training the machine leaning models can involve providing a set of real microscopy sequencing images of DNA or DNB array-based sequencing of a reference sequence. The set of real microscopy images are representative of a given sequencing process. That is, the set of real Images are representative of the specific machine or process that was used to generate them. For sufficient machine and/or process representation, images from multiple different instruments of the same type, from different DNA sequencing libraries and repeated runs of the same library per instrument may be used. This allows for training that is specialized for multiple different machines and processes.

Base calls associated with the set of real microscopy sequencing images can also be provided. The base calls may be determined using a mapping to the reference sequence or sequence barcodes. The trained machine learning model can then be generated by using the set of real microscopy images and the base calls as training data. DNA arrays and associated images can have spots that are occupied by either multiple DNBs or by no DNBs (e.g., no binding chemistry or no loaded DNB or a DNB with a few template copies). These spots may be trained for by setting the values of the training data to zero for no DNBs and a combination of base probabilities for mixed DNBs. This enables more accurate base calls and more accurate estimates of their probabilities. After training, the residual values of the output of the machine learning model for empty DNB spots can then be used to determine information regarding the background of the specific imaging system.

In an example, training data may involve realistic simulations and known sequences can be used as ground truth. The realistic simulations may be synthetic microscopy images, generated as described with respect to FIG. 1. Alternatively, real images may be used in the training data. The real images may be of PCR-free libraries from controlled genomes or from optionally barcoded synthetic DNA using known genomic sequences for DNA nanoballs. In another example, images generated from fully synthetic libraries where their sequence is fully known may be used. Sequences can be mapped that were generated using traditional base-calling methods, and the library sequence can be used for the mapped sequences to generate the accurate base-call output array for the corresponding real images.

Using accurate (e.g., 99.1%, 99.3%, 99.5%, 99.7%, 99.9% or higher) sequences of DNA nanoballs determined by mapping or barcodes for training can eliminate the need to determine accurate sequencing intensities in real low-resolution images. The machine learning model 1200 calling bases from images can replace individual image processing and base calling steps providing computational efficiency, adaptability by sufficient training to different nonuniformities and artifacts in real data including specific training for each sequencing system.

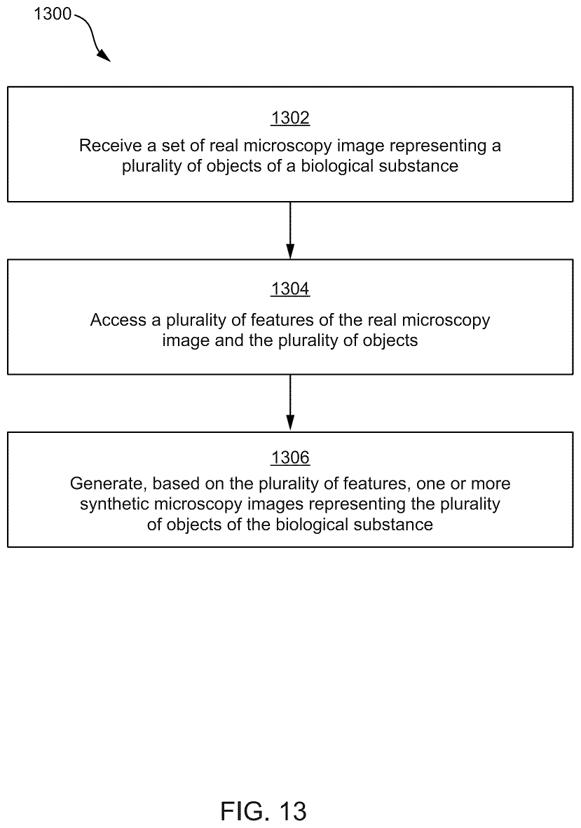

FIG. 13 is a flowchart of a method 1300 for generating synthetic microscopy images according to embodiments of the present disclosure. The processing depicted in FIG. 9 may be implemented in software (e.g., code, instructions, program) executed by one or more processing units (e.g., processors, cores) of the respective systems, hardware, or combinations thereof (e.g., the intelligent selection machine) described herein. The software may be stored on a non-transitory store medium (e.g., on a memory device). The method presented in FIG. 13 and described below is intended to be illustrative and non-limiting. Although FIG. 13 depicts the various processing steps occurring in a particular sequence or order, this is not intended to be limiting. In certain alternative embodiments, the steps may be performed in some different orders, or some steps may also be performed in parallel.

At block 1302, a set of real microscopy images representing a plurality of objects of a biological substance is received. The real microscopy images may show arrays of DNA nanoballs.

At block 1304, a plurality of features of the real microscopy image and the plurality of objects is accessed. The features may include physical characteristics of the objects and imaging characteristics of the real microscopy image. A simulation of sequencing biochemistry may be performed for the biological substance to generate a distribution related to the objects based on the features. In addition, a point spread function can be generated based on the features such that a seed intensity corresponding to a signal volume for each object can be determined.

At block 1306, one or more synthetic microscopy images representing the plurality of objects of the biological substance are generated based on the plurality of features. The synthetic microscopy images can substantially mimic the real microscopy images. The synthetic microscopy images can be generated based on the features and a seed image that is generated based on the seed intensities of the objects.

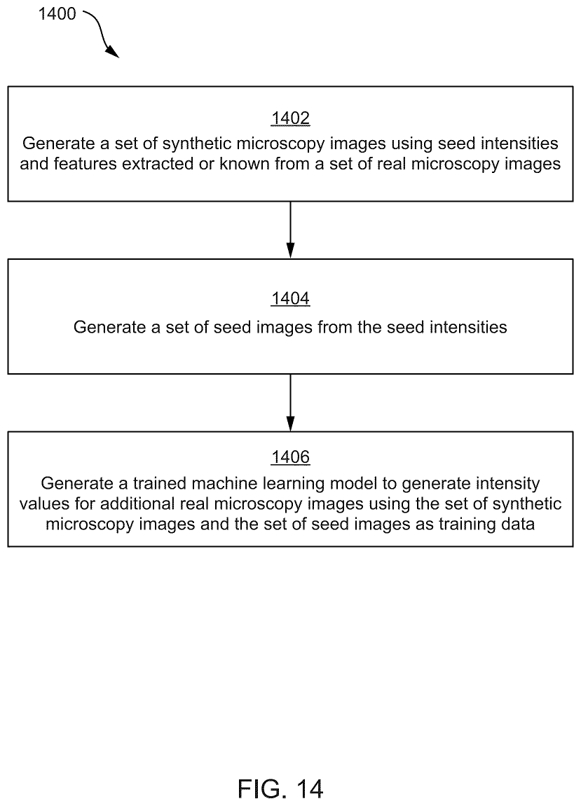

FIG. 14 is a flowchart of a method 1400 for developing an intensity extraction model according to embodiments of the present disclosure. The processing depicted in FIG. 14 may be implemented in software (e.g., code, instructions, program) executed by one or more processing units (e.g., processors, cores) of the respective systems, hardware, or combinations thereof (e.g., the intelligent selection machine) described herein. The software may be stored on a non-transitory store medium (e.g., on a memory device). The method presented in FIG. 14 and described below is intended to be illustrative and non-limiting. Although FIG. 14 depicts the various processing steps occurring in a particular sequence or order, this is not intended to be limiting. In certain alternative embodiments, the steps may be performed in some different orders, or some steps may also be performed in parallel.

At block 1402, a set of synthetic microscopy images is generated using seed intensities and features extracted or known from a set of real microscopy images. The synthetic microscopy images can be generated to mimic the set of real microscopy images. The synthetic microscopy images can be generated based on the features extracted and simulations of sequencing biology that generate the seed intensities.

At block 1404, a set of seed images is generated from the seed intensities. Each seed image can correspond to a synthetic microscopy image of the set of synthetic microscopy images. Each pixel in the seed image can represent a signal volume for an object.

At block 1406, a trained machine learning model is generated to generate intensity values for additional real microscopy images using the set of synthetic microscopy images and the set of seed images as training data. The set of synthetic microscopy images are used as input and the seed images are used as ground truths so that a machine learning model learns to generate intensity values for microscopy images. Real microscopy images can then be input into the trained machine learning model and intensity values for the real microscopy image can be output.

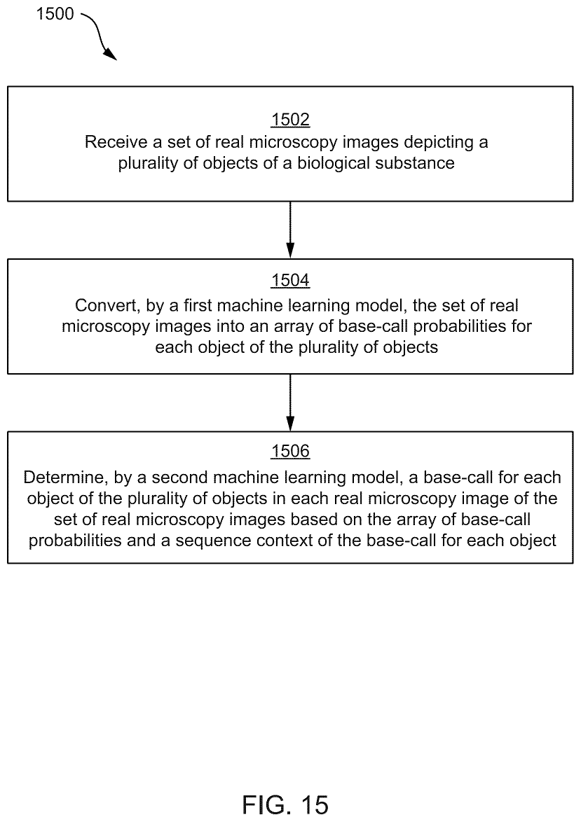

FIG. 15 is a flowchart of a method 1500 for using a base-calling model according to embodiments of the present disclosure. The processing depicted in FIG. 15 may be implemented in software (e.g., code, instructions, program) executed by one or more processing units (e.g., processors, cores) of the respective systems, hardware, or combinations thereof (e.g., the intelligent selection machine) described herein. The software may be stored on a non-transitory store medium (e.g., on a memory device). The method presented in FIG. 15 and described below is intended to be illustrative and non-limiting. Although FIG. 15 depicts the various processing steps occurring in a particular sequence or order, this is not intended to be limiting. In certain alternative embodiments, the steps may be performed in some different orders, or some steps may also be performed in parallel.

At block 1502, a set of real microscopy images depicting a plurality of objects of a biological substance is received. The set of real microscopy images correspond to different labeled bases in a DNA sequence. So, the set of real microscopy images may include four images. A first image can correspond to adenine, a second image can correspond to guanine, a third image can correspond to cytosine, and a fourth image can correspond to thymine.

At block 1504, a first machine learning model converts the set of real microscopy images into an array of base-call probabilities for each object of the plurality of objects. The first machine learning model can be a CNN that receives the set of real microscopy images as input. Multiple sets of real microscopy images for multiple cycles may be input into the first machine learning model. The arrays of base-call probabilities for each channel can be combined into combined base-call probability array.

At block 1506, a second machine learning model determines a base-call for each object of the plurality of objects in each real microscopy image of the set of real microscopy images based on the array of base-call probabilities and a sequence context (e.g., cycle number, gene positioning, etc.) of the base-call for each object. The second machine learning model may receive the combined base-call probability array as input. The second machine learning model can output a refined base-call probability array, from which the base-call for each object can be determined.

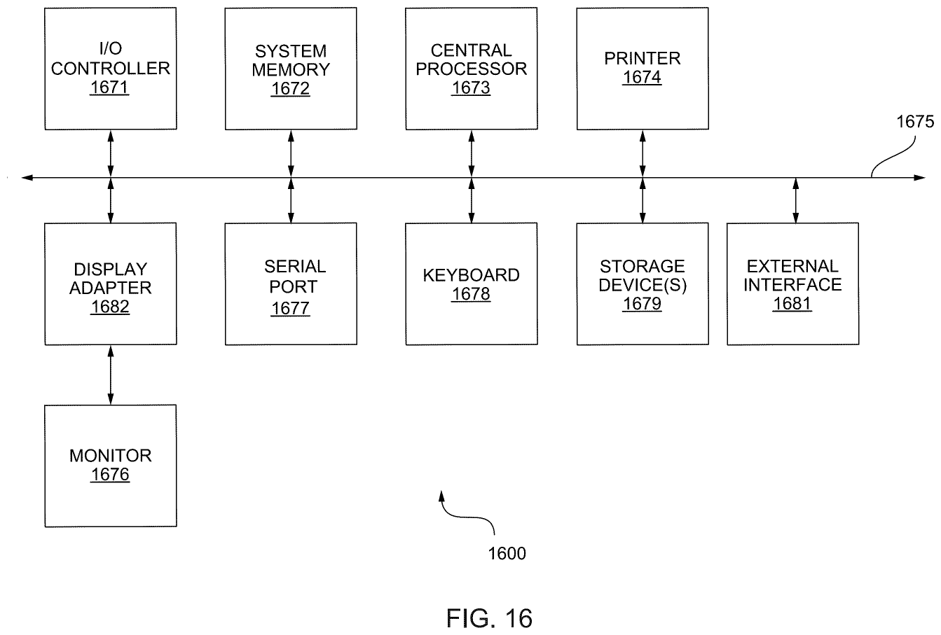

Any of the computer systems mentioned herein may utilize any suitable number of subsystems. Examples of such subsystems are shown in FIG. 16 in computer system 1600. In some embodiments, a computer system includes a single computer apparatus, where the subsystems can be the components of the computer apparatus. In other embodiments, a computer system can include multiple computer apparatuses, each being a subsystem, with internal components.

The subsystems shown in FIG. 16 are interconnected via a system bus 1675. Additional subsystems such as a printer 1674, keyboard 1678, storage device(s) 1679, monitor 1676, which is coupled to display adapter 1682, and others are shown. Peripherals and input/output (I/O) devices, which couple to I/O controller 1671, can be connected to the computer system by any number of means known in the art, such as serial port 1677. For example, serial port 1677 or external interface 1681 (e.g., Ethernet, Wi-Fi, etc.) can be used to connect computer system 1600 to a wide area network such as the Internet, a mouse input device, or a scanner. The interconnection via system bus 1675 allows the central processor 1673 to communicate with each subsystem and to control the execution of instructions from system memory 1672 or the storage device(s) 1679 (e.g., a fixed disk, such as a hard drive or optical disk), as well as the exchange of information between subsystems. The system memory 1672 and/or the storage device(s) 1679 may embody a computer readable medium. Any of the data mentioned herein can be output from one component to another component and can be output to the user.

A computer system can include a plurality of the same components or subsystems, e.g., connected together by external interface 1681 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

### Examples

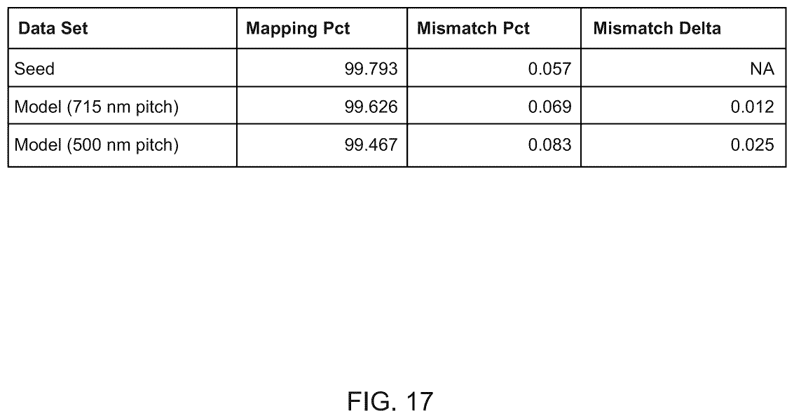

FIG. 17 shows example results of using a machine learning model for intensity extraction according to various embodiments. For the analysis, synthetic images were generated with a similar feature set to real data. Two machine learning models were trained for different DNA nanoball pitch spacings of a 715 nm pitch and a 500 nm pitch. The base calling performance on intensities extracted using the machine learning models for the top 95% DNA nanoballs by size for 500 cycles was evaluated. The base calling performance is expressed as mapping and mismatch percentages. The base calling performances for the machine learning models were compared with the base calling performance from the seed intensities, which corresponds to the perfect intensity extraction. Overall, it can be seen that the base calling performance is similar between the seed intensities and the intensities extracted using the machine learning models since there is only 0.025% difference in mismatch percentage between seed intensities and the machine learning model for the 500 nm pitch. This demonstrates the effectiveness of using machine learning models for intensity extraction.

### Additional Considerations

It should be understood that any of the embodiments of the present disclosure can be implemented in the form of control logic using hardware (e.g., an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C# or scripting language such as Perl or Python using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present disclosure may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of exemplary embodiments of the disclosure has been presented for the purposes of illustration and description. It is not intended to be exhaustive or to limit the disclosure to the precise form described, and many modifications and variations are possible in light of the teaching above. The embodiments were chosen and described in order to best explain the principles of the disclosure and its practical applications to thereby enable others skilled in the art to best utilize the disclosure in various embodiments and with various modifications as are suited to the particular use contemplated.

As used herein, when an action is “based on” something, this means the action is based at least in part on at least a part of the something. As used herein, the terms “similarly”, “substantially,” “approximately” and “about” are defined as being largely but not necessarily wholly what is specified (and include wholly what is specified) as understood by one of ordinary skill in the art. Further, the terms “similarly”, “substantially,” “approximately,” or “about” may be substituted with “within [a percentage] of” what is specified, where the percentage includes 0.1, 1, 5, and 10 percent. Further as used herein, a recitation of “a”, “an” or “the” is intended to mean “one or more” unless specifically indicated to the contrary.

1. A method comprising:
receiving a set of real microscopy images representing a plurality of objects of a biological substance, each object of the plurality of objects corresponding to one or more pixels of the set of real microscopy images;
accessing a plurality of features of the set of real microscopy images and the plurality of objects; and
generating, based on the plurality of features, one or more synthetic microscopy images representing the plurality of objects of the biological substance.

2. The method of claim 1, further comprising:
performing a simulation of sequencing biochemistry for the biological substance, wherein the simulation is configured to receive the plurality of features of a real microscopy image of the set of real microscopy images as an input; and
determining, based on the input, a seed intensity for each object of the plurality of objects of the real microscopy image, wherein the seed intensity corresponds to a signal volume for the object.

3. The method of claim 2, further comprising:
generating a seed image based on the seed intensity for each object of the plurality of objects, wherein each pixel in the seed image represents the signal volume for the object.

4. The method of claim 2, wherein generating the one or more synthetic microscopy images comprises:
generating a point spread function for the plurality of objects of the real microscopy image based on the plurality of features;
determining a signal distribution over a plurality of pixels by aggregating the point spread function and the seed intensity for each object; and
generating a synthetic image of the one or more synthetic microscopy images based on the signal distribution over the plurality of pixels and the plurality of features.

5. The method of claim 1, further comprising:
generating a trained machine learning model by training a machine learning model to generate intensity values for additional real microscopy images using the one or more synthetic microscopy images and a set of corresponding seed images as training data.

6. The method of claim 1, wherein the biological substance comprises a DNA array, an oligo array, a biological tissue, or an array of cells.

7. The method of claim 1, wherein the biological substance comprises a DNA array and the plurality of objects comprises a plurality of DNA nanoballs.

8. The method of claim 1, wherein the one or more synthetic microscopy images have substantially similar features to those of the set of real microscopy images.

9. A computer-program product tangibly embodied in a non-transitory machine-readable medium, including instructions configured to cause one or more data processors to:
generate a set of synthetic microscopy images using seed intensities and features extracted or known from a set of real microscopy images, each synthetic microscopy image representing a plurality of objects of a biological substance;
generate a set of seed images from the seed intensities, wherein each seed image corresponds to a synthetic microscopy image of the set of synthetic microscopy images, and wherein each pixel in the seed image represents a signal volume for an object of the plurality of objects; and
generate a trained machine learning model by training a machine learning model to generate intensity values for additional real microscopy images using the set of synthetic microscopy images and the set of seed images as training data.

10. The computer-program product of claim 9, further including instructions configured to cause the one or more data processors to:
input a real microscopy image into the trained machine learning model, the real microscopy image depicting an additional plurality of objects;
receive, from the trained machine learning model, an output representing a seed intensity for each object of the additional plurality of objects in the real microscopy image; and
generate a simulated microscopy image corresponding to the real microscopy image based on the output.

11. The computer-program product of claim 10, further including instructions configured to cause the one or more data processors to:
determine a difference between the real microscopy image and the simulated microscopy image.

12. The computer-program product of claim 11, further including instructions configured to cause the one or more data processors to:
in response to determining the difference, determine a set of features to use to generate subsequent simulated microscopy images.

13. The computer-program product of claim 11, wherein the trained machine learning model is a first trained machine learning model, and wherein the computer-program product further includes instructions configured to cause the one or more data processors to:
in response to determining the difference, input the simulated microscopy image into a second trained machine learning model; and
receive, from the second trained machine learning model, a result of an adjusted simulated microscopy image corresponding to the real microscopy image.

14. The computer-program product of claim 9, further including instructions configured to cause the one or more data processors to:
generate the set of synthetic microscopy images by:
receiving the set of real microscopy images representing the plurality of objects of the biological substance, each object of the plurality of objects corresponding to one or more pixels of the set of real microscopy images;
accessing a plurality of features of the set of real microscopy images and the plurality of objects; and
generating, based on the plurality of features, one or more synthetic microscopy images representing the plurality of objects of the biological substance.

15. The computer-program product of claim 14, further including instructions configured to cause the one or more data processors to:
performing a simulation of sequencing biochemistry for the biological substance, wherein the simulation is configured to receive the plurality of features of a real microscopy image of the set of real microscopy images as an input; and
determining, based on the input, a seed intensity for each object of the plurality of objects of the real microscopy image, wherein the seed intensity corresponds to the signal volume for the object.

16. The computer-program product of claim 15, further including instructions configured to cause the one or more data processors to:
generate a seed image based on the seed intensity for each object of the plurality of objects, wherein each pixel in the seed image represents the signal volume for the object.

17. The computer-program product of claim 15, wherein generating the one or more synthetic microscopy images comprises:
generating a point spread function for the plurality of objects of the real microscopy image based on the plurality of features;
determining a signal distribution over a plurality of pixels by aggregating the point spread function and the seed intensity for each object; and
generating a synthetic image of the one or more synthetic microscopy images based on the signal distribution over the plurality of pixels and the plurality of features.

18. The computer-program product of claim 9, wherein the biological substance comprises a DNA array, an oligo array, a biological tissue, or an array of cells.

19. The computer-program product of claim 9, wherein the biological substance comprises a DNA array and the plurality of objects comprises a plurality of DNA nanoballs.

20. A system comprising:
one or more data processors; and
a non-transitory computer readable medium storing instructions which, when executed on the one or more data processors, cause the one or more data processors to:
generate a set of synthetic microscopy images using seed intensities and features extracted or known from a set of real microscopy images, each synthetic microscopy image representing a plurality of objects of a biological substance;
generate a set of seed images from the seed intensities, wherein each seed image corresponds to a synthetic microscopy image of the set of synthetic microscopy images, and wherein each pixel in the seed image represents a signal volume for an object of the plurality of objects; and
generate a trained machine learning model by training a machine learning model to generate intensity values for additional real microscopy images using the set of synthetic microscopy images and the set of seed images as training data.
